(19) United States
(12) Patent Application Publication
(10) Pub. No.: US 2022/0039401 A1
(43) Pub. Date: Feb. 10, 2022

(54) Proofing Chamber for Proofing Dough

(71) Applicant: König Maschinen Gesellschaft m.b.H., Graz (AT)

(72) Inventors: Thomas Rieger, Lieboch (AT); Hannes Stelzer, Lannach (AT)

(21) Appl. No.: 17/275,260

(22) PCT Filed: Sep. 11, 2019
(86) PCT No.: PCT/AT2019/060298

§ 371 Date: Mar. 11, 2021

(30) Foreign Application Priority Data
Sep. 13, 2018 (AT) A 50784/2018

(51) Int. Cl. A21C 13/02; F24C 14/00; A61L 2/18; A61L 2/10

(52) U.S. Cl. CPC A21C 13/02; F24C 14/005; A61L 2202/17; A61L 2/10; A61L 2202/15; A61L 2/186

(57) ABSTRACT

A proofing chamber for proofing dough. An outer frame includes profiled frame sections running at an angle to the gravitational direction and encloses the interior of the proofing chamber. A number of covering panels are arranged on the outer frame, and a conveyor device runs through the interior of the proofing chamber. The conveyor device is designed to transport dough pieces through the proofing chamber. The conveyor device defines a number of transport planes arranged substantially horizontally or vertically, in particular horizontally or vertically. At least one profiled surface of the outer frame profiled sections which run at an angle to the gravitational direction and are arranged below the transport plane or the point of the transport plane arranged at the top in the gravitational direction is inclined in the direction of the interior.

### TECHNICAL FIELD

The present teaching relates to a proofing chamber, and/or a method for proofing dough.

### BACKGROUND

Known from the prior art are proofing chambers and/or facilities with proofing chambers from the baking industry, which are made up essentially of sheet metal and profiled sections. To keep the manufacturing of such proofing chambers as simple as possible and to execute the introduction of, for example, doors and connecting elements by simple means, the profiled sections in such proofing chambers are usually constructed vertically and bolted together. A disadvantage of known proofing chambers, however, is the fact that they are difficult to clean, because materials, for example flour, dirt and/or water, remain deposited on the vertically built profiles and/or that niches for bacteria and dirt or insects develop owing to the bolting of the frameworks.

### SUMMARY

It is therefore one object of the present teaching to resolve these known problems and to provide a proofing chamber that facilitates cleaning.

The present teaching fulfills this object with a proofing chamber for proofing dough comprising:

an outer frame, which includes framing profiles running at an angle to the gravitational direction and which encloses the interior of the proofing chamber,

a number of covering panels which are arranged on the outer frame,

a conveyor device running through the interior of the proofing chamber, wherein the conveyor device is designed to transport dough pieces through the proofing chamber and wherein the conveyor device defines a number of transport planes arranged essentially horizontally or vertically, in particular horizontally or vertically.

According to the present teaching, it is hereby provided that at least one profile surface of the profiled frame sections of the outer frame, which run at an angle to the gravitational direction, and are arranged below those transport planes or that point of the transport plane which is arranged at the topmost area in the gravitational direction, is inclined in the direction of the interior. This arrangement serves to assure that dirt and water found inside the proofing chamber flow off into the interior.

Running at an angle to the gravitational direction means in this context that the profiled frame sections are not constructed in the gravitational direction, that is, vertically, but rather are aligned, for example, at an angle of less than or equal to 90° to the gravitational direction. Owing to the inclination in the direction of the interior, little or no material or dirt remains on the shaped tubes in cleaning the proofing chamber, because, for example, flour residue or water found in the interior of the proofing chamber simply slides off the profiled surfaces of the inclined profiled frame sections or is washed away.

Transport planes in this context are understood to mean those planes running essentially horizontal or vertical, along which dough pieces from the conveyor device are transported approximately horizontally or vertically through the proofing chamber. All profiled frame sections which—with the transport planes horizontally aligned—are situated below the highest-placed transport plane when viewed in the gravitational direction, or—with vertically arranged transport planes—are below that point of the transport plane that, when viewed in the gravitational direction, is arranged in the highest area, are, with an inventive proofing chamber, inclined in the direction of the interior.

With proofing chambers that comprise a number of approximately vertical transport planes running parallel to one another, the highest point is understood to be the diversion at the highest point of the transport planes. With proofing chambers that comprise a number of approximately horizontal transport planes running approximately parallel to one another, the highest point is situated in each case in the highest transport plane, that is, that transport plane that is arranged highest in the gravitational direction, and thus the highest point in each case is understood to be the highest transport plane that is arranged highest in the gravitational direction.

An outer frame, in which advantageously especially few niches and openings exist in which dirt and insects can possibly accumulate, can be configured if the outer frame is designed as free of openings and in particular of boreholes.

To configure an especially stable outer frame for a proofing chamber, it can be foreseen that the profile frame sections are designed as shaped tubes and have a square or rectangular cross-section, four lengthwise sides and four lateral planes. Understood as lateral planes in this context are those planes of the shaped tubes that extend between the four lengthwise sides and together form the enclosing surface of the shaped tube.

A particularly stable outer frame can be configured for a proofing chamber if the latter is made up of shaped tubes running perpendicular and parallel to the gravitational direction. In this context it can be especially advantageous for cleaning the proofing chamber if the shaped tubes are aligned in such a way that, between the gravitational direction and the lateral planes, inclined in the direction of the interior, of the shaped tubes running perpendicular to the gravitational direction, an angle is formed that is smaller than 60°, in particular smaller than 45°, preferably smaller than 30°, and thus water and dirt flow out of the interior of the proofing chamber. The angle here designates in each case that acute angle formed between an imaginary perpendicular straight line through the respective formed tube and the lateral plane inclined in the direction of the interior or else several lateral planes of the shaped tube inclined in the direction of the interior. This inclination of the lateral planes ensures that advantageously no additional material accumulates on the shaped tubes of the outer frame or can be easily removed or washed off in cleaning.

The outer frame for a proofing chamber of this type can be constructed especially easily if a 45° angle is formed between the gravitational direction and the lateral surface, inclined in the direction of the interior, of the shaped tubes running perpendicular to the gravitational direction. In this context it can be especially advantageous for a simple connection of the shaped tubes with one another if, at the corners of the proofing chamber made up by three shaped tubes, in the shaped tubes running perpendicular to the gravitational direction, one of the diagonal planes parallel to the cross-section surface and the respective other diagonal plane is aligned perpendicular to the gravitational direction and the lengthwise sides of the shaped tubes each forms a right angle with one another. The cross-section plane here designates that surface produced when the enclosing surface jointly defined by the four lateral planes is viewed in cross-section. The two diagonal planes here connect corners of this defined cross-section plane that are opposite one another.

To provide a proofing chamber in whose outer frame no niches exist for, in particular, bacteria or dirt, it can be foreseen that the profiled frame sections, or in some cases the shaped tubes, of the outer frame are permanently form-locked together, in particular by soldering.

To ensure an especially flexible application of a proofing chamber, for instance for the maximum number of dough varieties and dough consistencies, it can be foreseen that the conveyor device is configured as a plate- or belt-type conveyor device or as a conveyor device on which fermentation material carriers, in particular dough cups, are arranged for receiving dough pieces. Typical carriers of fermentation material, which find application in fermentation chambers, include examples such as dough cups.

Undesired release of liquid from the fermentation chamber or undesired penetration of liquid, dirt or insects into the fermentation chamber can advantageously be avoided if the covering panels are arranged on the outer frame in such a way that a closed outer enclosure is formed around the interior of the fermentation chamber with a floor, a ceiling opposite the floor and lateral walls, so that the interior of the fermentation chamber is liquid-tight.

Especially simple production of an inventive fermentation chamber with simultaneous securing of efficient cleaning can be ensured if the shaped tubes of the external frame running perpendicular to the gravitational direction are aligned so that a lengthwise edge in the gravitational direction is more distant from the base than the other three lengthwise edges of the particular shaped tube. This means, for example, that shaped tubes with a square cross-section are inclined or rotated in the direction of the interior in such a way that, for instance, a diagonal plane of the particular shaped tube runs perpendicular and a diagonal surface runs in the gravitational direction, so that two lengthwise edges run at the same distance in the gravitational direction from the base of the outer enclosure. The two other lengthwise edges, which are connected in the cross-section through the diagonal surface running parallel to the gravitational direction, run in this case, one at a greater distance and one at a lesser distance measured from the base of the outer enclosure in the gravitational direction.

This alignment of the diagonal surfaces of the shaped tubes, however, is not at all compulsory, and the advantageous effect is also obtained if the diagonal surfaces are oriented in a different way to the gravitational direction, whereby one of the four lengthwise edges is further removed from the base of the outer enclosure than the other three lengthwise edges of the particular shaped tube.

Also advantageous for efficient cleaning of an inventive fermenting chamber, simultaneously with simple configuration of the outer frame, can be to align the shaped tubes running parallel to the gravitational direction in such a way that in each case two of their lateral planes are facing the interior of the fermentation chamber.

To configure a, for example, liquid-tight sealed outer enclosure that can be removed easily from the outer frame for thorough cleaning and simultaneously leaves no niches in which dirt can accumulate, it can be foreseen that the ceiling-covering panels that constitute the ceiling and the shaped tubes of the outer frame are reversibly materially bound together.

It can be particularly advantageous in this respect that the ceiling-covering panels have support surfaces adjusted to at least one, preferably two lateral surfaces of each of the shaped tubes, because the ceiling-covering panels in this case, without forming niches, are contiguous with the outer frame and the shaped tubes and moreover are removable without tools. An outer enclosure, which successfully makes do with a particularly low number of ceiling-covering panels, is obtained when the ceiling-covering panels in each case are contiguous with at least two shaped tubes of the outer frame.

An inventive fermentation chamber can provide an outer enclosure that is especially simple to clean and simultaneously easily removable if the base-covering panels that constitute the base and the shaped tubes of the outer frame are reversibly materially bound together and the base-covering panels in each case are contiguous with at least two shaped tubes of the outer frame or can be introduced into guide rails in or below the outer frame. It can be particularly advantageous in this respect if the base-covering panels have at least one outer wall adjusted in its inclination to a lateral plane of the shaped tubes and preferably at least one handle strip running along the outer wall, because such base-covering panels can also serve, for example, as a flour collection tray, which can be easily pushed below the outer frame, for example, removed again, and cleaned.

To guarantee simple access to the interior of the fermentation chamber, for example for maintenance or cleaning tasks, it can be foreseen that the side-covering panels that form the lateral walls are arranged so that they can be pivoted onto the shaped tubes of the outer frame. To ensure simple structure of such side-covering panels, for example, for cleaning tasks, it can be foreseen that the side-covering panels can be introduced by means of shaped tubes running preferably in tube holes perpendicular to the gravitational direction or are arranged on hinges on the outer frame fastened to suspensions welded to the shaped tubes.

To prevent unintentional opening of the side-covering panels, it can be foreseen that the side-covering panels can be pivoted out of a closed position, in which the side-covering panels are contiguous with at least two shaped tubes of the outer frame and that at least one switch, which is designed to detect any pivoting of the side-covering panels out of the closed position, is arranged on the side-covering panels. This means that the switch determines if a side-covering panel is opened, wherein it can also be foreseen that in this case, for example, a warning signal is given, so that the operating person is alerted that a side-covering panel of the fermentation chamber is opened.

To assure that also brackets for operating elements of the conveyor device running in the fermentation chamber, such as chains, sprockets or shafts, are easy to clean, it can be foreseen that, on the frame profiles, in some cases on shaped tubes, of the outer frame, brackets are arranged reversibly materially bound, in particularly bolted. This configuration of the brackets advantageously ensures that the latter are accessible from all sides for cleaning tasks.

The present teaching further relates to a fermentation chamber with a conveyor device that includes a belt or chain gear with a belt or chain system and a tensing device for self-sufficient tensing of the belt or chain system. According to the present teaching, it is foreseen here for simple cleaning of the belt or chain system that the tensing device includes at least a tensing cylinder with a piston that can move inside it. The tensing device here is arranged between the outer frame and a belt disc or a chainwheel of the belt or chain system so that upon force modification in the belt or chain system, the belt disc or chainwheel can be displaced in relation to the outer belt. Such an arrangement of the tensing device assures advantageously that the belt or chain system can easily be removed for cleaning of the fermentation chamber and then can be effortlessly re-installed without requiring complex tensing by hand of the belt or chain system.

Particularly simple cleaning of the tensing device can be achieved if the tensing device is arranged on the outer frame, in particular on the brackets mounted on the outer frame, for the operating elements of the conveyor device.

Simple cleaning of the interior of the fermentation chamber to remove dirt such as adhering flour, or efficient self-cleaning of the fermentation chamber, can be assured if a cleaning device for cleaning the interior is provided, which includes at least a water-dispensing device, particularly one arranged on the outer frame, and/or at least a drainage opening, wherein the drainage opening is configured in the base-covering panels that form the base of the outer enclosure. For instance, water nozzles can be arranged on the outer frame or the ceiling-covering panels, which dispense water for cleaning tasks on the fermentation chamber, so that dirt is rinsed away and flows out through the at least one drainage opening in the base.

Efficient cleaning of the fermentation material carriers is obtained if a cleaning station for cleaning fermentation material carriers is provided, which is arranged in the interior of the fermentation chamber or in an additional housing connected to the interior and includes at least one suction device and/or brushes and/or at least one water-dispensing device. To assure automated transport of the fermentation material carriers into the cleaning station and thus to avoid manual expansion, it can be foreseen here that the conveyor device is configured to transport the fermentation material carriers through the cleaning station.

In addition to cleaning of the fermentation material carriers, to provide a drying and sterilization means for the fermentation material carriers, it can be foreseen that the cleaning station includes a drying device for drying the fermentation material carriers, in particular by means of infrared radiation and/or a disinfection device for disinfecting fermentation material carriers. Particularly efficient disinfection of the fermentation material carriers can be achieved if the disinfection device includes at least a device for applying hydrogen peroxide to the fermentation material carrier and/or at least an illumination means for radiating dough traces with ultraviolet light.

An additional aspect of the present teaching foresees providing a method for cleaning a fermentation chamber. Also proposed by the present teaching is that at least one profile surface of the profiled frame sections running at an angle to the gravitational direction of the outer frame is inclined in the direction of the interior and that the dirt left on at least one profile surface is washed off with the cleaning device, wherein water left on the profile surface and/or cleaning substances and/or dirt runs off into the interior owing to the inclination of the profile surface, in particular completely.

Additional advantages and configurations of the present teaching can be seen from the description and the accompanying drawings.

The present teaching is schematically depicted hereinafter by means of particularly advantageous embodiments, which are not, however, to be understood as restrictive, and shown schematically in the drawings and described by way of example with reference to said drawings.

### BRIEF DESCRIPTION OF THE DRAWINGS

FIG. 1 shows a frontal view of an embodiment of an inventive fermentation chamber.

FIG. 2 shows an overhead view of the inventive fermentation chamber.

FIG. 3 shows a cross-section through the embodiment.

FIG. 4 shows a side view of the embodiment.

FIG. 5 shows a diagonal view of the embodiment.

FIG. 6 shows a detail view of a section depiction in the area of the ceiling.

FIG. 7 shows a hinge for securing the side-covering panels.

FIG. 7a shows a second embodiment of a hinge for affixing the side-covering panel.

FIG. 8 shows a longitudinal section through the embodiment.

FIG. 9 shows a diagonal view of the outer frame.

FIG. 10 shows a base-covering panel in detail.

FIG. 11 shows a detail view of a ceiling-covering panel.

FIG. 12 shows a detail view of the tensing device in built-in condition.

FIG. 13 shows a detail view of a side-covering panel.

FIG. 14 shows a detail view of a corner of the first embodiment.

### DETAILED DESCRIPTION

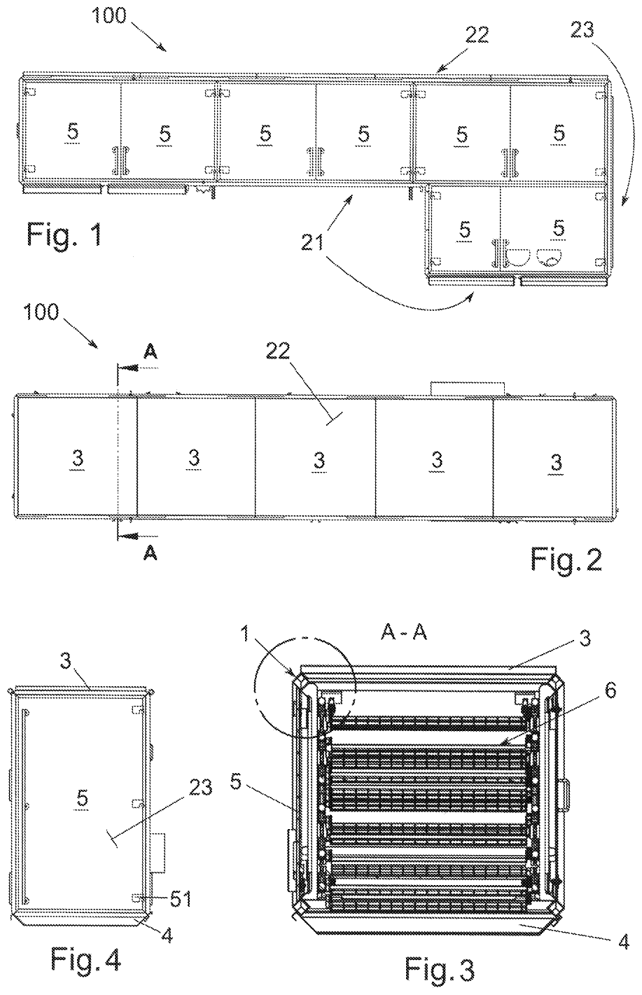

FIGS. 1, 2, 4 and 5 each show an embodiment of an inventive fermentation chamber 100 for fermenting dough, which comprises a square base surface and a likewise rectangular cross-section surface. The fermentation chamber 100 includes an outer frame 10, which encloses an interior 30 of the fermentation chamber (FIG. 9) and comprises profiled frame sections running in the gravitational direction or at an angle to the gravitational angle, and, in this embodiment, consist of shaped tubes 1, as shown in detail in FIG. 9. The outer frame 10 in the embodiment is made up of two individual areas or modules, each with rectilinear cross-section and base surfaces. Owing to the structure made of shaped tubes 1, the outer frame 10 can easily be expanded with the addition of additional modules of this type, in which various devices for additional dough-processing steps can be housed.

In addition, the fermentation chamber 100 includes a conveyor device 6, on which in the illustrated embodiment fermentation material carriers 7 configured as dough cups are arranged for receiving dough portions. Alternatively, the conveyor device 6 can be configured as a plate or belt conveyor device.

The conveyor device 6 in the illustrated embodiment includes several horizontally aligned transport planes, along which pieces of dough are transported through the fermentation chamber 100. Alternatively, the conveyor device 6 of an inventive fermentation chamber 100 can also include a number of vertically aligned transport planes.

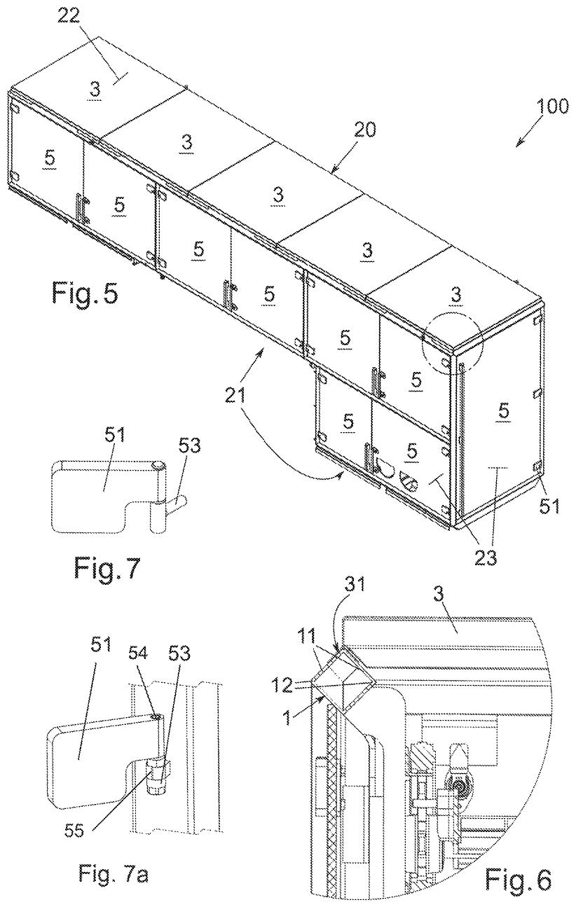

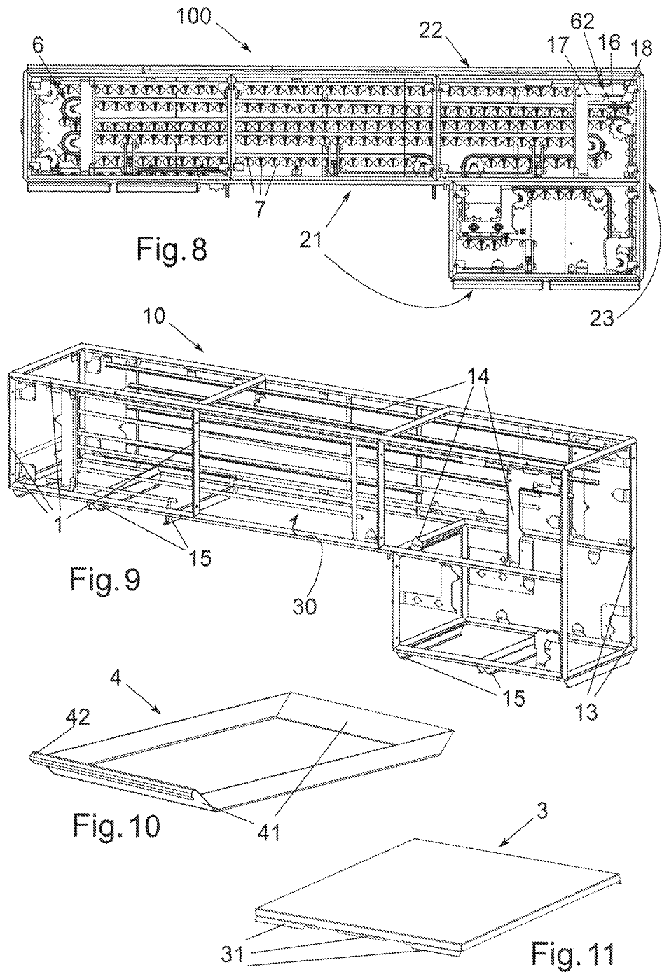

As can be seen in particular in FIG. 1 or FIG. 5, the outer frame 10 in the illustrated embodiment includes two areas, so that advantageously dough portions in the fermentation material carriers 7 are not only transported through the interior 30 by the conveyor device 6 for fermentation or leaving dough to stand, as is shown in the left-hand or upper area of the fermentation chamber 100 in FIG. 8. Thanks to the expansion of the interior 30 by a second outer-frame module, also additional dough-processing steps, such as lengthwise rolling in the interior 30 of the fermentation chamber 100, are performed in the illustrated embodiment.

The inventive fermentation chamber 100 further includes a number of covering panels 3, 4, 5, arranged on the outer frame 10, which together form a closed outer enclosure 20.

The fermentation chamber 100, in addition, comprises a conveyor device 6 running in the interior 30, on which fermentation material carriers 7 are arranged for receiving dough portions, as shown in greater detail FIG. 8 or FIG. 3. The conveyor device 6 is designed to transport the fermentation material carriers 7 through the fermentation chamber 100.

Optionally the inventive fermentation chamber 100 can also include means to generate a stable atmosphere in the interior 30 of the fermentation chamber 100 with controlled air humidity and temperature conditions conducive to the fermentation of dough.

The profiled frame sections of the outer frame 10 of the inventive fermentation chamber operate in such a way that at least one profile plane of each profiled frame section aligned with the gravitational direction is inclined in the direction of the interior 30 in such a way that dirt and water inside the fermentation chamber 100 flow into the interior 30. As can be seen in FIG. 9, the profiled frame sections in the illustrated embodiment are configured as shaped tubes 1 and have a square cross-section as well as four lengthwise edges 12 and four lateral planes 11, as depicted more fully in the detail view of FIG. 6.

In the illustrated embodiment, the outer frame 10 is made up of shaped tubes running perpendicular and parallel to the gravitational direction. The shaped tubes running perpendicular to the gravitational direction are rotated or inclined at a 45° angle to the direction of the interior 30. As shown in FIG. 6, this means that, inclined in the direction of the interior 30, the two lateral planes 11 of each shaped tube 1 running perpendicular to the gravitational direction form a 45° acute angle with the gravitational direction.

It can also be seen in FIG. 6 that, in the illustrated embodiment, the diagonal planes of the cross-section surface of the shaped tubes 1 perpendicular to the gravitational direction run parallel or perpendicular to the gravitational direction, so that one of the lengthwise edges 12 is farther away from the base than the other three lengthwise edges 12. FIG. 14 shows a detail view of a corner of the outer frame 10 of the inventive fermentation chamber 100, on which three shaped tubes 1 are connected with one another in such a way that two lateral planes 11 of the shaped tube 1 running parallel to the gravitational direction are oriented into the interior 30 of the fermentation chamber 100 and the diagonal planes stand parallel or perpendicular to the gravitational direction.

Alternatively, those profiled frame sections or shaped tubes 1 of the outer frame 10—which run at an angle to the gravitational direction and are arranged above those transport planes or that point of the transport plane that is arranged highest in the gravitational direction—can also run vertically, for example, rather than inclined in the direction of the interior 30.

This configuration of the outer frame 10, advantageously, assures that, while the fermentation material carriers 7 are transported through the fermentation chamber 100 by means of the conveyor device 6, flour, for example, which is scattered from the fermentation material carriers 7, does not remain stuck to the shaped tubes 1, but instead glides directly into the interior 30 of the fermentation chamber 100 or can be easily rinsed off the shaped tubes 1 during cleaning of the fermentation chamber 100 with water

In any of the connecting areas between the shaped tubes 1 of the outer frame 10, to avoid the formation of niches, in which, for example, refuse such as flour can accumulate or possibly vermin can infiltrate, the shaped tubes 1 in the embodiment are welded together. Alternatively, the shaped tubes may also be materially connected together in other ways.

The outer enclosure 20 of the fermentation chamber 100 illustrated in FIGS. 1, 2, 4 and 5 is constituted by arranging a number of covering panels 3, 4, 5 on the outer frame 10, so that the outer enclosure 20 is configured as enclosing the interior 30 of the fermentation chamber 100. The outer enclosure 20 includes a base 21, lateral walls 23 and a ceiling 22 opposite the base 21. The external enclosure 20 closes off the interior 30 of the fermentation chamber 100 in fluid-tight manner, so that neither liquid from the interior 30 can emerge nor undesired moisture can penetrate into it.

The base 21 of the outer enclosure 20 in the illustrated embodiment is formed of square or rectangular base-covering panels 4 made of sheet metal, which are reversibly materially connected with the shaped tubes 1 of the outer frame 10. In the illustrated embodiment, the base-covering panels 4 are contiguous in each case with at least two shaped tubes 1 of the outer frame 10 or can be inserted into guide rails 15 under the outer frame 10.

The inserted base-covering panels 4 in the illustrated embodiment consist of base-covering panels 4 configured as flour collection trays, which comprise outer walls 41 adjusted in their inclination to two lateral planes 11 of the shaped tubes 1 and a handle strip 42 running along an outer wall 41. These base-covering panels 4 are advantageously configured so that the lateral walls shaped in the guide rails 15 run vertically, while the lateral walls 41 contiguous with the profiled frame sections are slanted, so that dirt sliding off the outer frame 10 is easily caught in the base-covering panel 4 and collected there. Because these base-covering panels 4 can be inserted under the outer frame 10, during a maintenance or cleaning procedure they can easily be withdrawn and dirt caught in them can be removed.

The ceiling 22 of the outer enclosure 20 in the illustrated embodiment is made up of square ceiling-covering panels 3 of sheet metal, which are reversibly materially connected with the shaped tubes 1 of the outer frame 10. Alternatively, the ceiling-covering panels 3, for example, can be of rectilinear configuration. This material connection is achieved if the ceiling-covering panels 3 have support surfaces 31 which are adjusted to both lateral planes 11 of the particular shaped tube 1, with which the ceiling-covering panel 3 is contiguous. The materially bound connection between a ceiling-covering panel 3 and a shaped tube 1 is shown in detail in FIG. 6 and assures that the ceiling-covering panels 3 can be removed without requiring tools for the purpose. Alternatively, the ceiling-covering panels 3 can be mounted in reversible material connection, for example by means of insertion of locking pins in the shaped tube 1 on the outer frame 10.

The configuration of the ceiling-covering panel 3 with support surfaces 31 assures that, owing to the inclination of the shaped tube 1 in the direction of the interior 30, no niches develop in which dirt can be deposited, as would be the case with currently available ceiling-covering panels 3. In addition, owing to the materially bound connection, the ceiling-covering panels 3 are connected firmly enough with the outer frame 10 so that the latter cannot accidentally slip off the shaped tubes 1. Simultaneously it is assured that these are removable without tools and can be removed easily from the shaped tubes 1, for instance to perform cleaning and maintenance tasks or to exchange the ceiling-covering panels 3.

The lateral walls 23 of the outer enclosure 20 in the illustrated embodiment are made up of square or rectangular side-covering panels 5 of sheet metal, which are arranged to be pivoted onto the shaped tubes 1 of the outer frame 10. Alternatively, the side-covering panels 5 can also be constructed, for example, of plexiglass, allowing the course of the processing steps in the interior 30 of the fermenting chamber 100 to be observed if the lateral walls 23 are closed.

In the outer frame 10 in the shaped tubes 1 running parallel to the gravitational direction, bore holes 13 (FIG. 9) are arranged, which serve to affix the side-covering panels 5 on the outer frame 10. For pivotable affixing of the side-covering panels 5, hinges 51 insertable in the bore holes 13 are mounted on the side-covering panels 5. For this purpose, the hinges 51 have an extension 53, which in each case can be introduced into a bore hole 13 in a shaped tube 1 of the outer frame 10. Simultaneously, thereby, a sufficiently firm fastening of the side-covering panels 5 is assured on the outer frame 10 as well as easy pivoting of the side-covering panels 5, while at the same time it is a simple matter to exchange the side-covering panels 5 by means of easy withdrawal of the hinges from the outer frame 10. The hinges 51 further assure that the side-covering panels 5 can be opened wide in order to allow easy access to the interior 30 of the fermentation chamber 100.

Alternatively, as shown in FIG. 7a, the side-covering panels 5 can be arranged on the outer frame 10 by means of hinges 51 and in this manner provide doors, whereby the hinges 51 are suspended in a stud. The stud 54 is introduced into a suspension 55, which is welded onto the outer frame 10 and protected by means of a nut from falling out. The stud 54 is of two-part configuration, wherein the first part is arranged in the suspension 55 and stands on the second part of the hinge 51. The second part is arranged concentrically with the first part, so that the distance of the hinge 51 or the respective side-covering panel 5 affixed to the hinge 51 can be modified by rotating the stud.

This offers the additional advantage that the outer frame 10 in this case is equipped completely free of borings and thus fewer niches are present in which dirt can possibly accumulate.

The side-covering panels 5 in the illustrated embodiment are situated on at least two shaped tubes 1 of the outer frame 10 and comprise a switch 52 that is configured to indicate whether a contact prevails between the side-covering panel 5 and the shaped tube 1 and in this way to detect whether the side-covering panels pivot out of a closed position, in which the latter are contiguous with at least two shaped tubes 1 of the outer frame 10. If pivoting of the side-covering panels 5 is detected by the switch 52, then it is possible, for example, that the detection information can be transmitted to a control center from where the fermentation chamber 100 is operated, or for example if an optical or acoustical warning signal is conveyed to the user of the fermentation chamber 100. This assures that unintentional opening of the outer enclosure 20 of the fermentation chamber 100 is quickly determined and corrected.

As can be seen in FIG. 8, the conveyor device 6 in the area of the base 21 of the outer enclosure 20 enters the interior 30 of the fermentation chamber 100 and is driven by a chain gear with a chain system 61. Along the lengthwise extension of the fermentation chamber 100, the chain system 61 here is moved back and forth several times via chain wheels 18 lengthwise in the interior 30, so that the fermentation materials 7 arranged on the conveyor device 6 remain for a given time period in the interior 30 of the fermentation chamber 100 or are transported inside it. The fermentation materials 7 here are arranged perpendicular to the transport direction of the conveyor device 6 to allow several dough portions to be incorporated in one dough cup 7.

To assure ideal fermentation of the dough, in an inventive fermentation chamber, means for producing constant moist air and temperature conditions, such as a heating apparatus or air-conditioning unit and water-spraying devices can be provided in the interior 30.

Brackets 14 for the drive elements of the conveyor device 6 are reversibly materially bound on the shaped tubes 1 of the outer frame 10 in the illustrated embodiment. The brackets 14 in the embodiment are bolted to the outer frame 10 in such a way that they are accessible and can be cleaned from all sides.

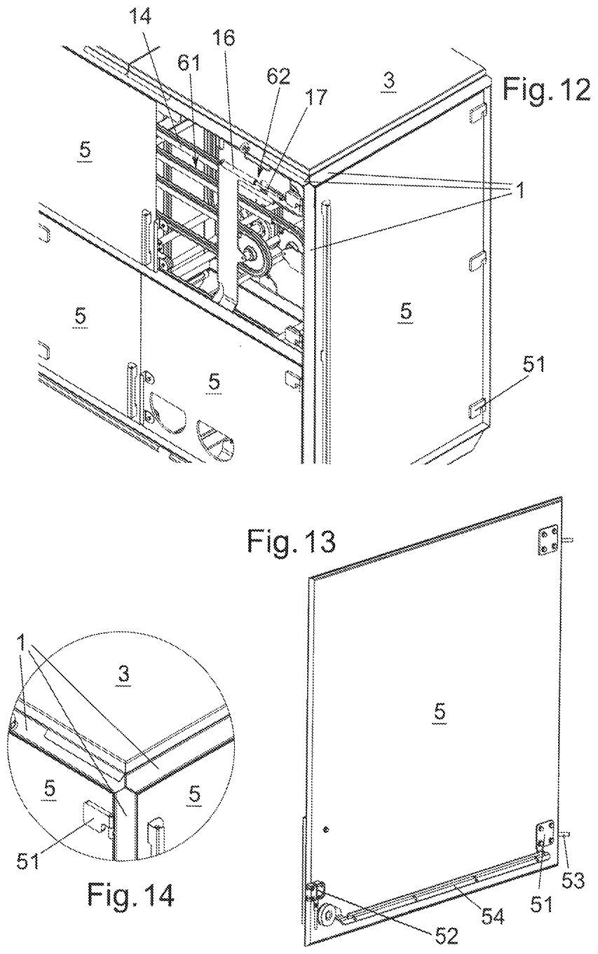

For simple dismantling and cleaning of the chain system 61 of the conveyor device 6, the conveyor device 6 in the illustrated embodiment includes a tensing device 62, which independently tenses the chain system 61. An embodiment of an inventive tensing device 62, shown in FIGS. 8 and 12, includes a tensing cylinder 16 with a piston that can move in it. The piston 17 is arranged between the outer frame 10 and a chain wheel 18 of the chain system 61 in such a way that, upon force modification in the chain system 61, the chain wheel is displaced with respect to the outer frame 10. This can be achieved, for instance, if in the interior of the cylinder 16 a constant or predetermined pressure prevails, which for example lets the piston 17 move out of position if the chain system 61 has too little tension and the chain system 61 is re-stressed. Thus, the chain system 61 can easily be removed for cleaning of the fermentation chamber 100 and be cleansed of dirt. Thereafter it can easily be arranged on the brackets 14 again without requiring laborious re-tensing by hand. The spanning device 62 in the illustrated embodiment is arranged on the brackets 14 for the drive elements of the conveyor device 6 on the outer frame 10.

Alternatively, the tensing device 62 can also take the form of a spring, for example as gas pressure spring, in which a constant or predetermined gas pressure prevails, so that the chain system 61 is re-tensed independently by the gas pressure spring if the chain system 61 loses tension.

In all embodiments of an inventive fermentation chamber 100, a cleaning unit for cleaning the interior 30, using an inventive method, can be foreseen in order to avoid laborious hand cleaning of the interior 30. The cleaning unit here can include water-dispensing devices on the outer frame 10 and/or at least a drainage opening in the base 21 of the outer enclosure 20. It is possible, for example, using such a cleaning unit, while operating the fermentation chamber or in given cleaning intervals, to remove dirt that possibly adheres in the interior 30 of the fermentation chamber 100, so that the dirt simply flows away through the run-off opening in the base 21. For this purpose, water nozzles, for example, are activated in the interior 10 of the fermentation chamber 100 to dispense water to the interior 30 so that flour and dough residues are liquefied and run off from the inclined shaped tubes of the outer frame 10 or the outer enclosure 20 or the conveyor device 6.

Since, owing to the configuration of the outer frame 10 of the fermentation chamber 100 made of profiled frame sections or formed tubes 1, the latter, as described before, is especially simple to expand, a cleaning station for cleaning of the fermentation material carriers 7 can be foreseen in all embodiments of an inventive fermentation chamber 100. The cleaning station here can be housed in the interior 30 of the fermentation chamber 100 or can be arranged in additional housing adjoining the interior 30. To avoid hand expansion of the fermentation material carriers 7 or removal of the conveyor device 6, the conveyor device 6 can be operated by the cleaning station arranged in the interior 30 or else the cleaning station adjoining the fermentation chamber 100 and can be configured to transport the fermentation material carriers 7 through the cleaning station. Thus, it can be foreseen that the conveyor device 6 is transported by the cleaning station at given cleaning intervals or else in ongoing operation of the dough cups 7.

The cleaning station here can include at least a suction device and/or brushes and/or at least a water dispensing device. Thus, it is advantageously possible to clean the fermentation material carriers 7 mechanically by means of brushes, and addition or alternatively to conduct suction cleaning of the dirt. In addition, or alternatively thereto, wet cleaning of the fermentation goods carriers 7 can be conducted by water jet or rotating water nozzles.

The cleaning station, in addition, can include a drying device for drying of fermentation material carriers 7, wherein the drying for example can be conducted by means of infrared radiation or else hot air nozzles. Optionally the cleaning station, in addition, can include an antibacterial device to disinfect the fermentation material carriers 7, which at least can include a device for applying hydrogen peroxide to the material carriers 7 and, in addition or alternatively, illuminating means for radiating the fermentation material carriers 7 with ultraviolet light. By means of such a treatment of the fermentation material carriers 7, they are then germ-free and ready for refilling with dough portions.

A particularly preferred embodiment of the fermentation chamber foresees that the outer frame 10 and the profiled frame sections are free of bore holes and other holes or recesses, but rather are smoothly configured. This prevents dirt from collecting in niches or holes where mold formation or bacterial herds develop. All elements affixed on the outer frame 10 are fastened by adhesive connections, which are glued upon the profiled frame sections or fixed to the outer frame 10 by welding.

1. A proofing chamber for proofing dough, comprising:
an outer frame which includes profiled frame sections running at an angle to the gravitational direction and which encloses the interior of the proofing chamber;
a number of covering panels which are arranged on the outer frame; and
a conveyor device which runs through the interior of the proofing chamber;
wherein the conveyor device is designed to transport dough pieces through the proofing chamber, and whereby the conveyor device defines a number of transport planes arranged substantially horizontally or vertically;
wherein at least one profiled surface of the outer frame profiled sections which run at an angle to the gravitational direction and are arranged below the transport plane or the point of the transport plane arranged at the top in the gravitational direction is inclined in the direction of the interior.

2. The proofing chamber according to claim 1, wherein the outer frame is configured free of openings.

3. The proofing chamber according to claim 1, wherein the profiled frame sections are designed as shaping tubes and include one square or rectangular cross-section, four lengthwise edges and four lateral planes.

4. The proofing chamber according to claim 3, wherein the outer frame is made up of shaped tubes running perpendicular and parallel to the gravitational direction and between the gravitational direction and the lateral planes inclined in the direction of the interior, the formed tubes running perpendicular to the gravitational direction form an angle of less than 60°.

5. The proofing chamber according to claim 3, wherein, between the gravitational direction and the lateral planes, inclined in the direction of the interior, of the shaped tubes running perpendicular to the gravitational direction, an angle of 45° is formed, and
that, at the corners of the fermentation chamber formed in each case by three shaped tubes, in the shaped tubes running perpendicular to the gravitational direction, in each case one of the diagonal planes of the cross-section surface is directed parallel to the gravitational direction and the respective other diagonal plane perpendicular to the gravitational direction, and the lengthwise edges of the shaped tubes each form a right angle with one another.

6. The proofing chamber according to claim 1, wherein the profiled frame sections, in some cases shaped tubes, of the outer frame are permanently materially bound together.

7. The proofing chamber according to claim 1, wherein the conveyor device is configured as a plate or belt conveyor device or as a conveyor device on which a fermentation material carrier is arranged for receiving dough pieces.

8. The proofing chamber according to claim 1, wherein the covering panels are arranged on the outer frame in such a way that a closed outer enclosure is formed around the interior of the fermentation chamber with a base, a cover opposite the base and lateral walls, so that the interior of the fermentation chamber is watertight.

9. The proofing chamber according to claim 8, wherein the shaped tubes of the outer frame running perpendicular to the gravitational direction are aligned in such a way that one lengthwise edge is farther removed from the base in the gravitational direction than the other three lengthwise edges of the respective shaped tube, and/or
that the shaped tubes running parallel to the gravitational direction are aligned in such a way that in each case two of their lateral planes are facing the interior of the fermentation chamber.

10. The fermentation chamber according to claim 8, wherein the base-covering panels, which shape the cover, and the shaped tubes of the outer frame are reversibly materially bound together, wherein the ceiling-covering panels on at least one lateral plane of each of the shaped tubes have adjusted support surfaces and wherein it is provided that the ceiling-covering panels in each case are contiguous with at least two shaped tubes of the outer frame.

11. The fermentation chamber according to claim 10, wherein the base-covering panels, which form the base, and the shaped tubes of the outer frame are reversibly materially bound together, wherein the base-covering panels in each case are contiguous with at least two shaped tubes of the outer frame or can be introduced into guide rails in or under the outer frame and wherein the base-covering panels have at least one outer wall adjusted in its inclination to a lateral surface of the shaped tubes.

12. The fermentation chamber according to claim 10, wherein the lateral covering panels forming the lateral walls can be pivoted upward on the shaped tubes of the outer frame by hinges insertable in bore holes in shaped tubes running preferably perpendicular to the gravitational direction or on suspensions soldered onto the shaped tubes.

13. The fermentation chamber according to claim 11, wherein the lateral covering panels can be pivoted out of a closed position, in which the lateral covering panels are contiguous with at least two shaped tubes of the outer frame, and that in each case at least one switch is arranged which is designed to detect any pivoting of the lateral covering panels out of the closed position.

14. The fermentation chamber according to claim 10, wherein on the profiled framed sections, in some cases on the shaped tubes, of the outer frame, brackets for drive elements of the conveyor device are arranged and reversibly materially bound together.

15. The fermentation chamber according to claim 10, wherein the conveyor device includes a belt or chain gear with a belt or chain system and a tensing device for self-sufficient tensing of the belt or chain system,
wherein the tensing device is configured so that, upon a change in forces in the belt or chain system, the belt disc or chain wheel is adjustable in relation to the outer frame,
wherein the spanning device includes at least a tensing cylinder with a piston that is movable in the piston, wherein the piston is arranged between the outer frame and a belt disc or a chain wheel of the belt or chain system, or
is configured as a spring.

16. The fermentation chamber according to claim 15, wherein the tensing device is arranged on the outer frame.

17. The fermentation chamber according to claim 10, wherein a cleaning device for cleaning the interior is provided which includes, arranged on the outer frame, at least a water-dispensing device and/or at least a drainage opening, wherein the drainage opening is configured in the base-covering panels forming the base of the outer cladding.

18. The fermentation chamber according to claim 10, wherein a cleaning station for cleaning the fermentation material carriers is provided, wherein the cleaning station is arranged in the interior of the fermentation chamber or in an additional housing connected to the interior, whereby the cleaning station includes at least one suction device and/or brushes and/or at least one water dispensing device and wherein the conveyor device is configured to transport the fermentation material carriers through the cleaning station.

19. The fermentation chamber according to claim 18, wherein the cleaning station includes a drying device for drying the fermentation material carriers, and/or a disinfection device for disinfecting the fermentation material carriers, wherein the disinfection device includes at least one device for applying hydrogen peroxide to the fermentation material carriers and/or at least one illuminating means to radiate the fermentation material carriers with ultraviolet light.

20. A method for cleaning a fermentation chamber, comprising:
cleaning the fermentation chamber by a cleaning device,
wherein the fermentation chamber includes an outer frame, which includes profiled frame sections running at an angle to the gravitational direction and encloses an interior of the fermentation chamber,
wherein at least one profile plane of the profiled frame sections of the outer frame is inclined in the direction of the interior, and the dirt deposited on the at least one profiled surface is washed by the cleaning device, wherein water found on the profiled surface and/or cleaning material and/or dirt runs off into the interior owing to the inclination of the profiled surface.
